(19) United States
(12) Patent Application Publication
(10) Pub. No.: US 2007/0207542 A1
(43) Pub. Date: Sep. 6, 2007

(54) Manipulation of oxygen tension during in vitro follicle culture

(76) Inventors: Alan J. Russell, Gibsonia, PA (US); Matthew K. Heise, Pittsburgh, PA (US); Richard R. Koepsel, Pittsburgh, PA (US); Elizabeth A. McGee, Midlothian, VA (US)

Correspondence Address: Craig G. Cochenour, Esq.;Buchanan Ingersoll & Rooney PC, 301 Grant Street, 20th Floor, One Oxford Centre, Pittsburgh, PA, 15219, US

(21) Appl. No.: 11/713,201

(22) Filed: Mar. 2, 2007

Related U.S. Application Data
(60) Provisional application No. 60/779,346, filed on Mar. 3, 2006.

(51) Int. Cl. C12N 5/06

(52) U.S. Cl. 435/325

(57) ABSTRACT

A process for culturing an immature ovarian follicle in vitro is disclosed. The process includes providing a culture having one or more immature ovarian follicles and providing a sufficient amount of oxygen to the ovarian culture on an intermittent basis, stepwise basis, or continuously, or combinations thereof, for a sufficient amount of time over the duration of the culture period to effect the maturation of the immature ovarian follicle. Over the course of the culture period, the oxygen concentration is raised until, at the end of the culture period, the oxygen concentration is in the range preferably between about 3% by volume (22.8 mmHg) and about 20% by volume (152 mmHg).

### CROSS-REFERENCE TO RELATED APPLICATIONS

This Application claims the benefit of priority to pending U.S. Provisional Patent Application Ser. No. 60/779,346, filed on Mar. 3, 2006, entitled “Manipulation of Oxygen Tension During In vitro Follicle Culture, and having the same named inventors, namely, Alan J. Russell, Matthew K. Heise, Richard R. Koepsel, and Elizabeth A. McGee. U.S. Provisional Patent Application Ser. No. 60/779,346 is incorporated by reference into this Application as if fully rewritten herein.

### STATEMENT REGARDING FEDERALLY SPONSORED RESEARCH

The invention described herein was made in the course of work supported in part by the Women's Reproductive Health Research Center Program, NIH/NICHD 1K12HD38513. The Government may have certain rights in this invention.

### FIELD OF THE INVENTION

This invention is a process of oxygen manipulation for in vitro cultured ovarian follicles. Specifically, this process discloses regulating oxygen concentrations during in-vitro ovarian follicle culture over the duration of the culture period. The regulation of the oxygen concentration may be intermittently, in a stepwise fashion over time, continuously over a period of time, or combinations thereof, as described herein.

### BACKGROUND OF THE INVENTION

Therapeutic treatments for cancer and other diseases often adversely affect the reproductive cells of a patient. The very therapy that kills the cancer cells also kills the germ cells within the reproductive organs. For girls and women, this is a particularly harsh consequence because mammalian females are born with a limited supply of eggs. Should this original complement of eggs be destroyed, the result is sterility. For adult women, depletion of the supply of eggs results in menopause. Children whose eggs have been destroyed fail to undergo puberty. To a young girl, facing lifetime sterility as a result being cured of her cancer, a technique that would allow her to bear her own genetic children would be welcome indeed.

Recently many groups around the world have begun ovarian cryopreservation programs for young girls and women undergoing potentially sterilizing surgery or chemotherapy with the hope that follicles can be isolated from these tissues and grown in vitro at a later date. Since growing follicles do not generally survive freezing, only the very small primordial follicles remain viable after cryopreservation. Though follicles derived from mice can be grown up to maturity using conventional culture techniques, scientists have been unsuccessful with the in vitro development of follicles from species that have larger follicles, such as rats, pigs and humans. Intact rat and human preantral follicles have never been successfully cultured beyond the antral transition. However, follicles have traditionally been grown in standard incubators with atmospheric oxygen concentration. In vivo, preantral follicles exist in the relatively avascular cortex of the ovary. The oxygen available to these small follicles would have to diffuse from the peritoneal cavity or nearby large follicles that do have a blood supply. The partial pressure of oxygen in the peritoneal cavity has been measured at 40 mmHg a far cry from the ˜140 mmHg obtained in media exposed to normal incubator conditions. Further, the partial pressure of oxygen is expected to be even further reduced by crossing the ovarian capsule. Indeed the Po₂ across the sub-renal capsule drops from the peritoneal level of 40 mmHg to 14-19 mmHg. Therefore, the process of the present invention can be applied to these preserved ovarian follicles in order to better mimic the in vivo environment, aiding in the maturation of the follicles and, ultimately, restoring fertility to the patient. A second use of this process of the present invention is as a source of embryonic stem cells. Currently, the only sources of human embryonic stem cells are donated blastocysts from infertility treatments, blastocysts produced from donated gametes, and nuclear transfer. In each of these cases, the procedures involve the retrieval of female gametes for the purpose of fertilization. In a recent report from the Institute of Medicine, it states that “if the need for oocytes increases, it is possible that donations from clinical procedures or for nonfinancial motives may prove insufficient to meet the demands.” However, with this new process of the present invention, immature follicles obtained from cryopreserved ovarian tissue can be matured to yield viable oocytes for the production of embryonic stem cells. The process of the present invention therefore opens the door to a new pool of tissue as a source of oocytes without having to induce ovulation and invasively extract tissue from a female donor, a procedure that puts the donor at risk for serious complications such as severe ovarian hyperstimulation syndrome. The novel feature of this process of the present invention is the dynamic oxygen concentration protocol applied to in vitro cultured ovarian follicles. Existing in vitro procedures culture follicles at static oxygen concentrations. Traditionally, follicles have been cultured in standard incubators with atmospheric oxygen concentration. In recent years, there has been research studying the effects of lower oxygen concentrations on follicle development. In either case, the oxygen concentrations have remained constant for the duration of the culture periods.

### SUMMARY OF THE INVENTION

The present invention provides a process for culturing an immature ovarian follicle in vitro comprising providing a culture having one or more immature ovarian follicles, and providing a sufficient amount of oxygen to said ovarian culture over a sufficient period of time, in an intermittent fashion over a sufficient period of time, in a stepwise fashion over a sufficient period of time, continuously over a sufficient period of time, or combinations thereof, to effect the maturation of said immature ovarian follicle in an in vitro culture environment.

In another embodiment of the process of the present invention, as described herein, the immature ovarian follicles are selected from the group consisting of non-preserved ovarian follicles, preserved ovarian follicles, and vitrificated ovarian follicles.

Another embodiment of the process of the present invention provides for effecting the maturation of the immature ovarian follicles to yield viable oocytes for the production of embryonic stem cells.

In yet another embodiment of the process of the present invention, the process includes wherein the culture contains one or more of a primordial, primary, secondary, or antral follicles, or combinations thereof.

Another embodiment of the present invention includes providing the oxygen to the culture in a manner for creating an oxygen gradient throughout the in vitro culture period.

Several other embodiments of the process of the present invention include wherein the process includes providing the oxygen to the culture in a stepwise manner. This process may include wherein the stepwise manner comprises increasing the oxygen level. Further, this process may include decreasing the oxygen level. Yet further, the process may include one or more series of increasing and decreasing the oxygen levels during the in vitro culture period.

Another embodiment of the present invention, as described herein, provides including adding a sufficient amount of follicle stimulating hormone to the culture for enhancing the growth of the ovarian follicle culture. The follicle stimulating hormone may be a recombinant follicle stimulating hormone.

Other embodiments of the present invention include wherein the processes employ a culture containing one or more cumulus oocyte complexes, one or more intact ovarian follicles, or combinations thereof.

### BRIEF DESCRIPTION OF THE DRAWINGS

FIG. 1 shows a cultured rat oocyte with the first polar body extruded as indicated by the arrow.

### DETAILED DESCRIPTION OF THE INVENTION

As used herein the term “cryopreservation” means the process of freezing tissues for use at a later time, such process is well known by those skilled in the art. The term vitrification” describes the process of removing the water content from a tissue and then freezing the tissue for use at a later time, such process is also well known by those skilled in the art. The vitrification process is also known as a freeze drying process. The process of the present invention may be performed upon non-preserved immature ovarian follicle(s) and upon immature ovarian follicles that have previously been subjected to a cryopreservation or vitrification process.

One embodiment of this invention provides a process for culturing an immature ovarian follicle in vitro comprising providing a culture having one or more immature ovarian follicles and providing a sufficient amount of oxygen to the ovarian culture intermittently over a sufficient period of time, in a stepwise fashion over a period of time, or continuously over a sufficient period of time, or combinations thereof to effect maturation of said immature ovarian follicle in an in vitro culture environment. As used herein, the term “sufficient amount of time” means that period of time necessary to effect maturation of the immature ovarian follicle in the in vitro culture environment. A sufficient amount of time is, for example but not limited to, an effective amount of time ranging from about one (1) hour to about and including up to one hundred eighty (180) days. Preferably, a sufficient amount of time is from about three (3) to up to twenty one (21) days, and more preferably from about twelve (12) to about and including twenty four (24) hours. As used herein, the term “sufficient amount of oxygen” means an effective oxygen concentration ranging from about 0.01 percent (0.01%) to about twenty percent (20%) by volume to effect the maturation of the immature ovarian follicle in an in vitro culture environment.

In one embodiment of the present invention, the process concerns regulating oxygen concentrations during in-vitro ovarian follicle culture over the duration of the culture period. Oxygen concentrations of the immature ovarian follicle culture at the onset of the process generally, for example but not limited to, range from at about 0% (0 mmHg) to about 12% (91.2 mmHg) by volume. Over the course of the culture period, the present process raises the oxygen concentration until, at the end of the culture period, the effective oxygen concentration is in the range generally between from about three percent (22.8 mmHg) to about twenty percent (152 mmHg) by volume, and preferably the oxygen concentration ranges from about five percent (38 mmHg) to about twenty percent (152 mmHg) by volume. The oxygen concentrations are changed in an intermittent or stepwise fashion or in a continuous manner until the end of the culture period is reached.

An example of the instant process may be for a 20 day culture period wherein the culture has a beginning oxygen concentration of 3% (22.8 mmHg) by volume and comprises increasing the concentration about 1% (7.6 mmHg) by volume every two days (48 hours) until the culture ends on day 20 with an oxygen concentration in the incubator of about 12% (91.2 mmHg) to about 13% by volume. The advantage of the embodiments of the processes of the present invention over known follicle culture protocols that employ static oxygen concentrations is that, by increasing the oxygen concentrations in the incubator, it serves to mimic the dynamic in vivo oxygen concentrations to which in situ ovarian follicles are exposed. In vivo, preantral follicles exist in the avascular cortex of the ovary. As the follicle transitions into an antral follicle and continues to mature to a preovulatory follicle, it becomes vascularized, drastically increasing the oxygen available to the tissue. Therefore, in the general life span of a matured graafian follicle, the follicle experiences an increasing range of oxygen concentrations from initial concentrations that are virtually anoxic to having capillaries directly feeding the matured follicle.

In order to optimize any culture system, tissue engineering strives to recreate the tissue's natural environment in an in vitro setting. Conventional known culture systems employing a static oxygen concentration on cultured follicles fails to recreate the oxygen gradient present throughout the in vivo follicle development process. The role of oxygen or reactive oxygen species (ROS) on embryo development has been studied. It is believed that oxygen concentration in some animal species begin low and gradually increase with follicle vascularization but may then decrease in vivo with increasing biomass of the ganulosa cells. The ovulated egg is in the low O₂ intraperitoneal cavity, and then moves into the fallopian tubes and the uterus. It is known that the fallopian tube excretes many factors (of which many are yet undetermined) that likely support the oocyte/zygote. While not being bound by any specific theory, it is postulated that there are stage specific effects of oxygen on the developing oocyte depending upon the availability of protecting factors from the follicle or environment. There are differing patterns of mRNA expression in follicles grown at different oxygen (O₂) concentrations (ie. the oxygen concentration alters the transcriptome of the cultured follicle). Ultimately, egg quality depends upon normal folliculogenesis. A less differentiated follicle may not express all of the factors necessary to protect the oocyte from oxidative stress. It is postulated that this may account for the discrepancies that the Applicants have found in the literature regarding whether oxygen levels have a role in follicle culture. The present Applicants disclose a process for manipulating the oxygen tension during in vitro follicle culture for effecting the maturation of an immature ovarian follicle.

The present invention concerns the process of regulating the oxygen concentration during in-vitro ovarian follicle or cumulus oocyte complex culture over the duration of the culture period. Oxygen concentrations (by volume) at the onset of the culture are set between about 0 to 12%. Over the course of the culture period, the oxygen concentration is changed until, at the end of the culture period, the oxygen concentration is, preferably in the range of about 3% (22.8 mmHg) to about 20% (152 mmHg) oxygen by volume. The oxygen concentrations are changed intermittently or in a stepwise increasing manner or in a continuous manner, or a combination thereof, until the end of the culture period is reached. In another embodiment of this invention, the process provides for increasing the oxygen concentration as set forth herein, and then decreasing the oxygen concentration for a given period of time, then resuming to increase the oxygen concentration, and optionally repeating this cycle of increasing and decreasing the oxygen concentrations until a desired oxygen concentration is achieved for effecting the maturation of the immature ovarian follicles over the duration of the culture time period.

The following examples further illustrate the embodiments of the process of the present invention.

### EXAMPLES

### Example 1

### Oxygen Protocol for the Maturation of Immature Oocytes

### Animals and Ovarian Dissection

All animal experiments were performed in accordance with National Institutes of Health guidelines and with institutional approval. Sprague-Dawley rats were obtained from Hilltop Lab Animals (Pittsburgh, Pa.) and housed under standard conditions. The animals were sacrificed by CO₂ exposure and cervical dislocation. Ovaries were carefully dissected and placed immediately in warmed culture medium, consisting of Leibovitz L-15 Medium (Gibco BRL). The follicles were then mechanically dissected from the ovary using a pair of syringes with 26 gauge needles. All follicles used in the experiments were measured in two dimensions, using an inverted microscope fitted with an ocular micrometer.

### Follicle Culture

Culture media consisted of alpha Minimal Essential Medium (Gibco BRL, Invitrogen Corporation, Grand Island, N.Y.) with additives of 8-bromo-cGMP (5 mM), ITS+ (1% solution of insulin, 10 mg/L; transferrin, 5.5 mg/L; linoleic acid, 4.7 mg/L; selenium, 5 mg/L), Pen/Strep (1%, penicillin 100 U/ml, streptomycin 100 microgram/ml), all from Sigma Chemical Co. (St. Louis, Mo.), and recombinant Follicle Stimulating Hormone, rFSH, (Serono Laboratories, Geneva) in increasing increments from 0 IU/ml up to 100 IU/ml. Culture media was placed into 12×75 mm polypropylene culture test tubes (500 microliter/tube) and cultured in 5% CO₂ and 37° C. (Centigrade) humidified incubator. The oxygen level was increased throughout the culture period beginning at 2% (15.2 mmHg) and increased 1% (7.6 mmHg) every 48 hours until the end of the 17 day culture with a final oxygen level of 10% (76 mmHg).

Suspension culture was attained by placing the 6 ml (milliliter) culture tubes in a circular rotator plate (Glas-Col, Terre Haute, Ind.), having a diameter of 30.5 cm (centimeter), which was rotated around its horizontal axis at rate between 8-15 rpm (revolutions per minute). Therefore, as the plate rotates, the tubes slowly orbit the axis of the plate.

### Ovulation Induction and Oocvte Maturation

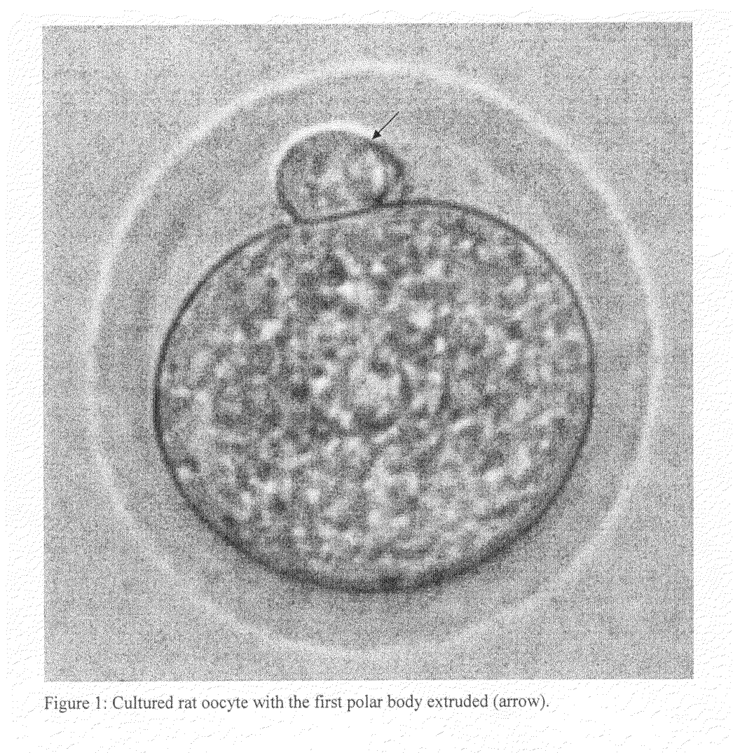

Follicles were removed from the culture media and placed into media containing alpha Minimal Essential Medium, Pen/Strep (1%, penicillin 100 U/ml, streptomycin 100 microgram/ml), and hCG (1.0 IU/mL) for 12 hours. Oocytes were then removed from their surrounding cumulus and placed into IVF-30 media (Vitrolife) supplemented with 30 mM NaCl. After 4 hours, oocytes were examined for the presence of an extruded polar body (FIG. 1).

### Example 2

### The Process of the Present Invention can be Applied to any Species

The process of Example 2 is essentially as described in Example 1 but the protocol does not have to be applied to only rats. The processes of the present invention may be applied to any agricultural animal—such as goats, cattle, pigs, yaks, horses, sheep,—or other species of interest such as dogs, cats, and primates including monkeys, apes, and humans. Furthermore, the process of the instant invention may be employed for producing gametes for endangered species.

### Example 3

### The Process can begin within a Range of Starting Oxygen Concentrations

The process of Example 3 is essentially as described in Example 1 but the protocol does not have a set starting oxygen concentration. In this embodiment of the process of the present invention, the process may start with oxygen concentrations ranging from 0% (0 mmHg) to 12% (91.2 mmHg).

### Example 4

### The Process Will End Within a Range of Ending Oxygen Concentration

The process of Example 4 is essentially as described in Example 1 but the protocol does not have a set ending oxygen concentration. In this embodiment of the present invention, the process ends with oxygen concentrations ranging from about 3% (22.8 mmHg) to about 20% (152 mmHg).

### Example 5

### The Duration Over Which this Process Takes Place Occurs Within a Range of Time Periods

The process of Example 5 is essentially as described in Example 1 but the protocol does not have a set time period for this process to be implemented. In this embodiment of the present invention, the process is applied over a time period ranging from about 1 hour to about 180 days. The time period for carrying out the process preferably shall be a sufficient time period in which to effect the maturation of the immature ovarian follicle. The time period for carrying out the process of the present invention shall vary dependent upon the species of a particular immature ovarian follicle.

### Example 6

### A Continuous, Stepwise, or Combination Method of Ramping the Oxygen Concentration Throughout the Culture Period can be Applied

In other embodiments of this invention, the present processes comprises essentially the process as is described in Example 1 but does not employ a ramping of the oxygen concentration intermittently throughout the culture period. The process comprises a continuous method of oxygen concentration ramping in which the oxygen concentration is constantly increasing at a set or at a variable rate for the duration of the culture period. For example, the oxygen level was increased throughout the culture period wherein the culture had a beginning oxygen concentration of 2% (15.2 mmHg) and wherein the oxygen concentration was increased at a rate of 1% (7.6 mmHg)/day until the end of a 10 day culture period with a resulting final oxygen level of 12% (91.2 mmHg).

Another embodiment of the process of the present invention provides a stepwise method of oxygen concentration ramping in which the oxygen concentration is changed at a time point and then held at a certain oxygen concentration for a period of time before being altered again or before the end of the culture period. For example, the oxygen level was increased throughout the culture period wherein the beginning oxygen concentration of the culture was at 2% (15.2 mmHg) and wherein the oxygen concentration was increased 1% (7.6 mmHg) every 48 hours until the end of the 17 day culture with a final resulting oxygen level of 10% (76 mmHg). This stepwise process of the instant invention may alter oxygen concentrations consistently, in which oxygen concentrations are all held for equal periods of time throughout the duration of the culture, or inconsistently, in which certain oxygen concentrations are held for longer periods of time than others.

Another embodiment of the present process provides a combination of the continuous and stepwise processes of oxygen concentration ramping in which between periods of stepwise holds at certain oxygen concentrations there are periods of continuous oxygen concentration ramping. For example, the oxygen level was increased throughout the culture period beginning at 3% (22.8 mmHg) and increased 1% (7.6 mmHg) every 48 hours for the first 6 days of the culture at which point the oxygen level is 6% (45.6 mmHg). Then, for the final 4 days of the culture period, the oxygen level was increased at a rate of 1.5% (11.4 mmHg)/day until the end of the 10 day culture period results in a final oxygen level of 12% (91.2 mmHg). The process may end with oxygen concentrations ranging from about 3% (22.8 mmHg) up to and including about 20% (152 mmHg).

### Example 7

### The Processes of Ramping Oxygen Concentrations can Both Increase and Decrease Oxygen Concentration Throughout the Culture Period

In another embodiment of the process of the present invention is as essentially as described in Example 5 but the ramping methods outlined in Example 6 do not only exclusively increase the oxygen concentration throughout the culture period. The oxygen concentrations can be exclusively increased or there can be series of increases and decreases throughout the culture period. The process may end with oxygen concentrations ranging from about 3% (22.8 mmHg) to about 20% (152 mmHg).

### Example 8

### The Processes are Applied to Follicles of any Stage

The processes of the present invention as essentially as described in Examples 1-7 may be applied to follicles existing at various stages including primordial, primary, secondary, and antral. The process can then end with the follicle in various stages including early antral, later antral, and pre ovulatory.

### Example 9

### The Processes can be Applied to Cumulus Oocvte Complexes

The processes of the present invention as essentially as described in Examples 1-7 may be applied to cumulus oocyte complexes as well as intact follicles.

It will be understood by those persons skilled in the art that the processes of the instant invention provide for the regulation of the oxygen concentration during in vitro ovarian follicle or cumulus oocyte complex culture.

Whereas particular embodiments of the instant invention have been described for purposes of illustration, it will be evident to those persons skilled in the art that numerous variations may be made without departing from the instant invention as defined in the appended claims.

What is claimed is:

1. A process for culturing an immature ovarian follicle in vitro comprising:
providing a culture having one or more immature ovarian follicles; and
providing a sufficient amount of oxygen to said ovarian culture intermittently, stepwise, or continuously, or combinations thereof, for a sufficient amount of time to effect maturation of said immature ovarian follicle in an in vitro culture environment.

2. The process of claim 1 including wherein said immature ovarian follicles are selected from the group consisting of non-preserved ovarian follicles, preserved ovarian follicles, and vitrificated ovarian follicles.

3. The process of claim 1 including effecting said maturation of said immature ovarian follicles to yield viable oocytes for the production of embryonic stem cells.

4. The process of claim 1 including wherein said culture contains one or more of a primordial, primary, secondary, or antral follicles, or combinations thereof.

5. The process of claim 1 including providing said oxygen to said culture in a manner for creating an oxygen gradient throughout said in vitro culture period.

6. The process of claim 1 including providing said oxygen to said culture in a stepwise manner.

7. The process of claim 7 including wherein said stepwise manner comprises increasing said oxygen level.

8. The process of claim 7 including wherein said stepwise manner comprises decreasing said oxygen level.

9. The process of claim 7 including wherein said stepwise manner comprises one or more series of increasing and decreasing said oxygen levels during said in vitro culture period.

10. The process of claim 1 including adding a sufficient amount of follicle stimulating hormone to said culture for enhancing the growth of said ovarian follicle culture.

11. The process of claim 10 including wherein said follicle stimulating hormone is a recombinant follicle stimulating hormone.

12. The process of claim 1 including wherein said culture contains one or more cumulus oocyte complexes.

13. The process of claim 1 including wherein said culture contains one or more intact ovarian follicles.

14. The process of claim 1 including wherein said oxygen is provided in an effective amount resulting in said culture having a final oxygen level at the end of the culture period ranging from about 3 percent to about 20 percent by volume.

15. The process of claim 1 including wherein said oxygen is provided to said culture for a period of time ranging from about one hour to about one hundred and eighty days.
